(19) United States
(12) Patent Application Publication
(10) Pub. No.: US 2013/0267836 A1
(43) Pub. Date: Oct. 10, 2013

(54) GUIDE PIN

(71) Applicants: Richard G. MAULDIN, Erie, CO (US); W. Carlton RECKLING, Cheyenne, WY (US); Scott A. YERBY, Montara, CA (US)

(72) Inventors: Richard G. MAULDIN, Erie, CO (US); W. Carlton RECKLING, Cheyenne, WY (US); Scott A. YERBY, Montara, CA (US)

(21) Appl. No.: 13/794,580

(22) Filed: Mar. 11, 2013

Related U.S. Application Data
(60) Provisional application No. 61/609,016, filed on Mar. 9, 2012.

(51) Int. Cl. A61B 17/88; A61B 6/00; A61B 6/12

(52) U.S. Cl. CPC A61B 17/8897; A61B 6/12; A61B 6/485
USPC 600/424; 606/96

(57) ABSTRACT

This application relates generally to guide pins and wires. More specifically, this application relates to guide pins and wires used in medical procedures such as bone fixation or fusion. One embodiment of the guide pin includes a distal end with a ball end cutter. Other embodiments of the guide pin include expandable prongs that can impede further advancement of the guide pin.

### CROSS REFERENCE TO RELATED APPLICATIONS

This application claims priority to U.S. Provisional Application No. 61/609,016, filed Mar. 9, 2012, titled “GUIDE PIN,” which is hereby incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference. For example, this application incorporates by reference in their entireties U.S. Patent Publication No. 2011/0087294 and U.S. Patent Publication No. 2011/0118785.

### FIELD

This application relates generally to guide pins and wires. More specifically, this application relates to guide pins and wires used in medical procedures such as bone fixation or fusion.

### BACKGROUND

Many types of hardware are available both for the fixation of bones that are fractured and for the fixation of bones that are to be fused (arthrodesed).

For example, the human hip girdle is made up of three large bones joined by three relatively immobile joints. One of the bones is called the sacrum and it lies at the bottom of the lumbar spine, where it connects with the L5 vertebra. The other two bones are commonly called “hip bones” and are technically referred to as the right ilium and-the left ilium. The sacrum connects with both hip bones at the sacroiliac joint (in shorthand, the SI-Joint).

The SI-Joint functions in the transmission of forces from the spine to the lower extremities, and vice-versa. The SI-Joint has been described as a pain generator for up to 22% of lower back pain.

To relieve pain generated from the SI Joint, sacroiliac joint fusion is typically indicated as surgical treatment, e.g., for degenerative sacroiliitis, inflammatory sacroiliitis, iatrogenic instability of the sacroiliac joint, osteitis condensans ilii, or traumatic fracture dislocation of the pelvis. Currently, screw and screws with plates are used for sacro-iliac fusion. At the same time the cartilage has to be removed from the “synovial joint” portion of the SI joint. This requires a large incision to approach the damaged, subluxed, dislocated, fractured, or degenerative joint.

To reduce soft tissue damage, a tissue dilator can be used to provide access to the surgical site. One common type of tissue dilator system includes a plurality of tubular sleeves of increasing diameter that are designed to slide over a guide pin or guide wire that has been inserted into the bone at the surgical site. As dilators of increasing diameters are sequentially slid over the guide pin, the tissue surrounding the guide pin is gradually pushed away from the guide pin, resulting in an opening in the tissue.

In addition, conventional guide pins have sharp, pointed tips that, in some cases, can be over-inserted, leading to impingement or damage of sensitive tissues, such as nerve tissue. Therefore, to further reduce the likelihood of soft tissue damage, an improved guide pin is desirable.

### SUMMARY OF THE DISCLOSURE

This application relates generally to guide pins and wires. More specifically, this application relates to guide pins and wires used in medical procedures such as bone fixation or fusion.

In some embodiments, a guide pin assembly is provided. The assembly includes a guide pin having a proximal end and a distal end, the distal end of the guide pin including a plurality of expandable prongs; and a sheath having an inner surface, a tapered distal end and an opening at the distal end, the sheath configured to cover at least a portion of the guide pin; wherein the expandable prongs are configured to be located within the sheath in a compressed state in a first configuration and are configured to be located at least partly outside the sheath in an expanded state in a second configuration.

In some embodiments, the guide pin has a shaft with threads that are engaged with corresponding grooves on the inner surface of the sheath, wherein the threads are configured to retract the sheath relative to the expandable prongs upon rotation of the guide pin relative to the sheath.

In some embodiments, the threads are pitched such that less than a half turn of the guide pin relative to the sheath is sufficient to fully deploy the expandable prongs.

In some embodiments, the guide pin is made of a shape memory metal alloy.

In some embodiments, the tapered distal end of the sheath has external threads.

In some embodiments, the tapered distal end of the sheath comprises a plurality of blade portions that are configured to be displaced outwards.

In some embodiments, the expandable prongs have a distal end that is sharpened.

In some embodiments, the distal end of the guide pin has a first beveled surface that extends past the distal end of the sheath which has a second beveled surface, wherein the first beveled surface and the second beveled surface are aligned.

In some embodiments, the first beveled surface and the second beveled surface have the same bevel angle.

In some embodiments, a guide pin assembly is provided. The assembly includes a guide pin having a proximal end and a distal end and an elongate body, the distal end of the guide pin including an expanded tip; a sheath having an expandable distal end with at least one slit, the sheath having a channel covering at least the elongate body of the guide pin, the channel having a non-expanded configuration with a first diameter and an expanded configuration with a second diameter; wherein the expanded tip has a diameter greater than the first diameter of the channel.

In some embodiments, the expanded tip of the guide pin extends past the distal end of the sheath when the channel is in the non-expanded configuration.

In some embodiments, the expanded tip of the guide pin is disposed within the sheath when the channel is in the non-expanded configuration.

In some embodiments, the expandable distal end of the sheath is configured to be expanded when the expanded tip of the guide pin is retracted within the channel of the sheath.

In some embodiments, a guide pin assembly is provided. The assembly includes a guide pin having a proximal portion and a distal portion, the distal portion comprising a coiled wire having a distal end that is sharp and pointed; a sheath that is reversibly disposed over at least the distal portion of the guide pin such that the coiled wire is in a compact configuration when the sheath is disposed over distal portion and an expanded configuration when the coiled wire is advanced out of the sheath, wherein the expanded configuration of the coiled wire has a larger diameter than the sheath.

In some embodiments, the guide pin is made of a superelastic metal alloy.

In some embodiments, the guide pin is configured to be advanced and retracted relative to the sheath by rotation of the guide pin relative to the sheath.

In some embodiments, a guide pin assembly is provided. The assembly includes a guide pin having a proximal portion and a distal portion, the distal portion comprising a plurality of wires that are twisted together in a compact configuration having a pointed tip; and a sheath that is reversibly disposed over at least the distal portion of the guide pin such that the plurality of wires are in the compact configuration when the sheath is disposed over distal portion and an expanded configuration when the plurality of wires are advanced past the sheath, the sheath having a proximal end and a distal end, wherein the expanded configuration of the plurality of wires has a larger diameter than the sheath.

In some embodiments, the proximal end of the sheath is covered by a removable cap.

In some embodiments, the proximal portion of the guide pin has a smaller diameter than the distal portion such that a transverse shoulder portion provides a transition from the proximal portion to the distal portion, wherein the shoulder portion is configured to engage a complementary abutment within the sheath to align the pointed tip of the plurality of wires in the compact configuration with the distal end of the sheath.

In some embodiments, a guide pin for drilling into bone is provided. The guide pin includes an elongate shaft having a proximal end and a distal end, the distal end being blunt and rounded and having at least one cut-out with a cutting edge extending proximally from the distal end.

In some embodiments, the cut-out forms a helical or spiral flutes.

In some embodiments, the cut-out has a flat surface that is joined with a curved surface.

In some embodiments, a method of inserting a guide pin into bone is provided. The method includes drilling a guide pin through bone to a desired location in a gradual and controlled manner, the guide pin having a blunt and rounded distal end and at least one cutting edge extending from the distal end; and viewing the location of the guide pin using fluoroscopy.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of an implant structure.

FIGS. 2A-2D are side section views of the formation of a broached bore in bone according to one embodiment of the invention.

FIGS. 2E and 2F illustrate the assembly of a soft tissue protector system for placement over a guide wire.

FIGS. 3 and 4 are, respectively, anterior and posterior anatomic views of the human hip girdle comprising the sacrum and the hip bones (the right ilium, and the left ilium), the sacrum being connected with both hip bones at the sacroiliac joint (in shorthand, the SI-Joint).

FIGS. 5 to 7A and 7B are anatomic views showing, respectively, a pre-implanted perspective, implanted perspective, implanted anterior view, and implanted cranio-caudal section view, the implantation of three implant structures for the fixation of the SI-Joint using a lateral approach through the ilium, the SI-Joint, and into the sacrum.

FIGS. 8A-8C illustrate one embodiment of a guide pin that can be used with a variety of implants and surgical procedures.

FIGS. 9A-9D illustrate another embodiment of a guide pin with a feature that impedes or stops guide pin advancement.

FIGS. 9E-9G illustrate another embodiment of a guide pin with prongs covered by a guide pin sheath.

FIGS. 10A-10D illustrate other embodiments of a guide pin assembly that include a feature to reversibly stop or impede guide pin assembly advancement.

FIGS. 11A-11C illustrate another embodiment of a guide pin that includes an expandable braided wire tip.

### DETAILED DESCRIPTION

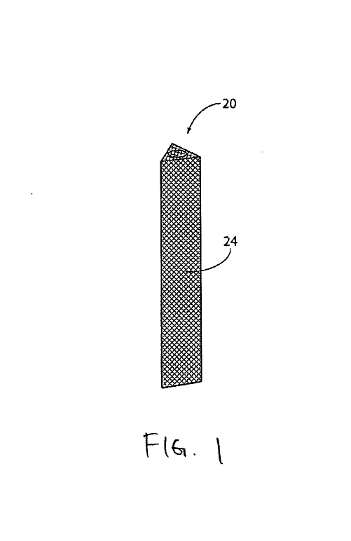

Elongated, stem-like implant structures 20 like that shown in FIG. 1 make possible the fixation of the SI-Joint (shown in anterior and posterior views, respectively, in FIGS. 3 and 4) in a minimally invasive manner. These implant structures 20 can be effectively implanted through the use of a lateral surgical approach. The procedure is desirably aided by conventional lateral and/or anterior-posterior (A-P) visualization techniques, e.g., using X-ray image intensifiers such as a C-arms or fluoroscopes to produce a live image feed that is displayed on a TV screen.

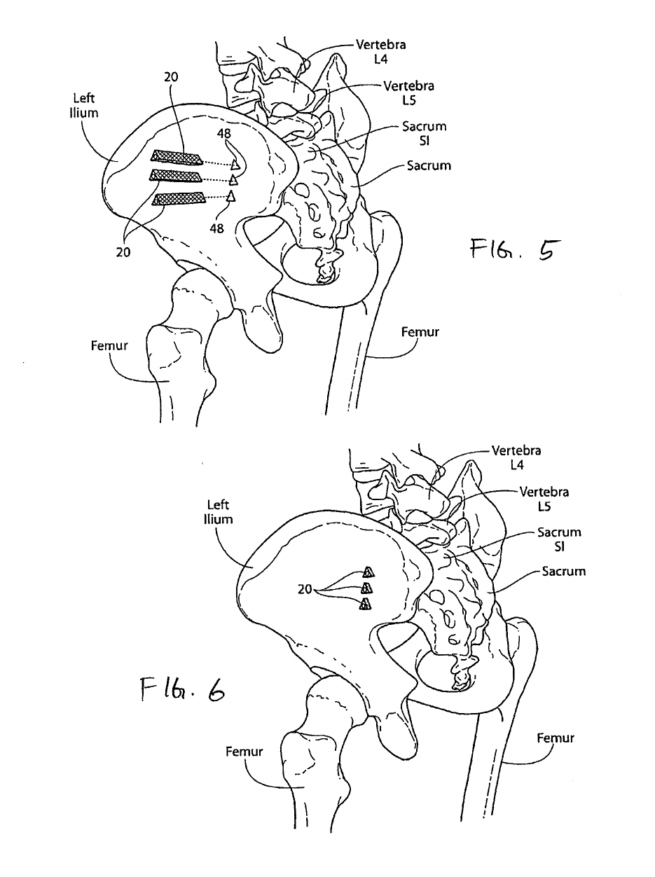

In one embodiment of a lateral approach (see FIGS. 5, 6, and 7A/B), one or more implant structures 20 are introduced laterally through the ilium, the SI-Joint, and into the sacrum. This path and resulting placement of the implant structures 20 are best shown in FIGS. 6 and 7A/B. In the illustrated embodiment, three implant structures 20 are placed in this manner. Also in the illustrated embodiment, the implant structures 20 are rectilinear in cross section and triangular in this case, but it should be appreciated that implant structures 20 of other cross sections can be used. For example, the implant structures can have a square cross-section. In some embodiments, the implant structures can have a curvilinear cross-section, such as circular, oval or elliptical. The cross-sections discussed above refer to the transverse cross-section of the implant rather than a longitudinal cross-section taken along the longitudinal axis of the implant structure. In addition, the term rectilinear describes a device that is defined or substantially defined by straight lines. This includes, for example, triangles, squares, and other polygons, and also includes triangles, squares and other polygons having rounded corners. In contrast, the term curvilinear is meant to describe devices that are defined by only curved lines, such as a circle or ellipse, for example.

Before undertaking a lateral implantation procedure, the physician identifies the SI-Joint segments that are to be fixated or fused (arthrodesed) using, e.g., the FABER Test, or CT-guided injection, or X-ray/MRI of SI Joint.

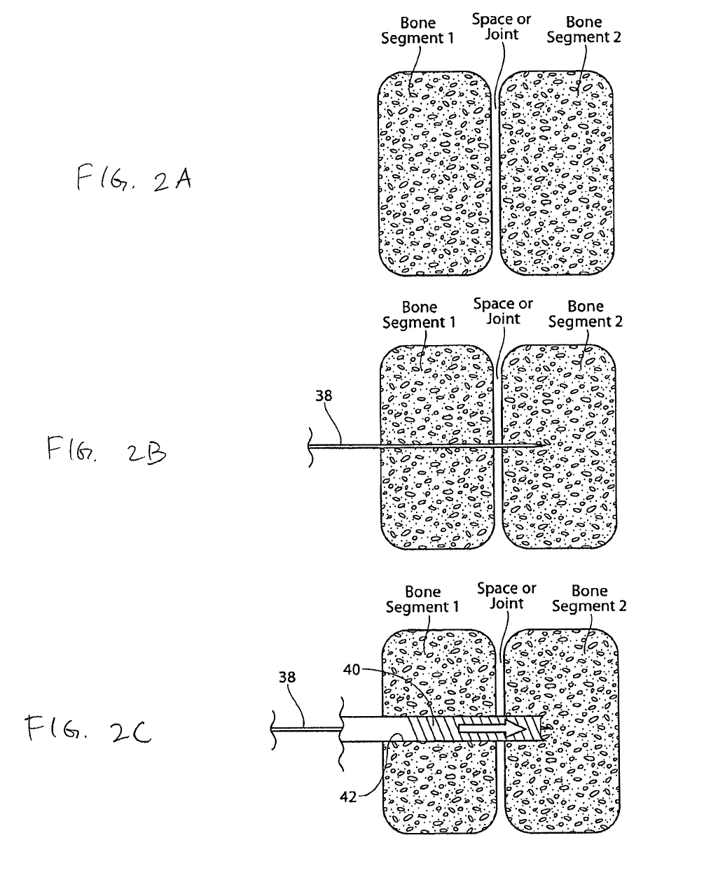

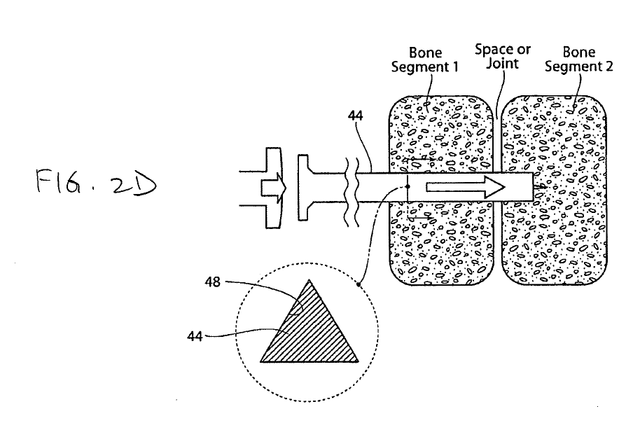

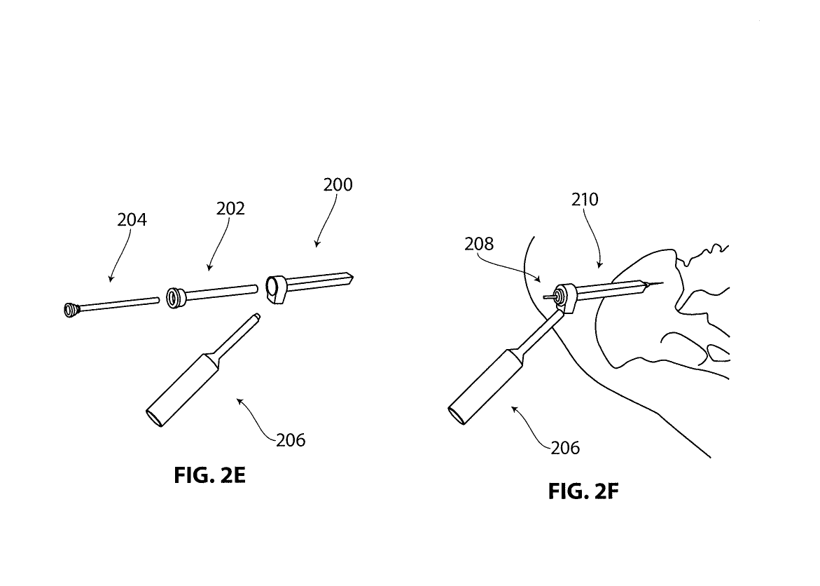

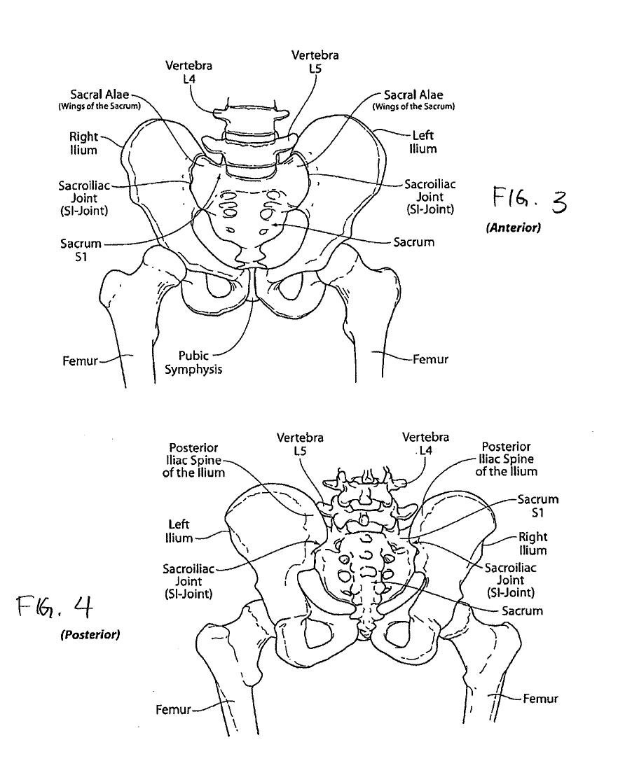

Aided by lateral and anterior-posterior (A-P) c-arms, and with the patient lying in a prone position, the physician aligns the greater sciatic notches and then the alae (using lateral visualization) to provide a true lateral position. A 3 cm incision is made starting aligned with the posterior cortex of the sacral canal, followed by blunt tissue separation to the ilium. From the lateral view, the guide pin 38 (with sleeve (not shown)) (e.g., a Steinmann Pin) is started resting on the ilium at a position inferior to the sacrum end plate and just anterior to the sacral canal. In A-P and lateral views, the guide pin 38 should be parallel to the sacrum end plate at a shallow angle anterior (e.g., 15 to 20 degrees off horizontal, as FIG. 7B shows). In a lateral view, the guide pin 38 should be posterior to the sacrum anterior wall. In the A-P view, the guide pin 38 should not violate the sacral foramina. This corresponds generally to the sequence shown diagrammatically in FIGS. 2A and 2B. A soft tissue protector (not shown) is desirably slipped over the guide pin 38 and firmly against the ilium before removing the guide pin sleeve (not shown).

Over the guide pin 38 (and through the soft tissue protector), the pilot bore 42 is drilled in the manner previously described, as is diagrammatically shown in FIG. 2C. The pilot bore 42 extends through the ilium, through the SI-Joint, and into the sacrum. The drill bit 40 is then removed.

The shaped broach 44 is tapped into the pilot bore 42 over the guide pin 38 (and through the soft tissue protector) to create a broached bore 48 with the desired profile for the implant structure 20, which, in the illustrated embodiment, is triangular. This generally corresponds to the sequence shown diagrammatically in FIG. 2D. The triangular profile of the broached bore 48 is also shown in FIG. 5.

FIGS. 2E and 2F illustrate an embodiment of the assembly of a soft tissue protector or dilator or delivery sleeve 200 with a drill sleeve 202, a guide pin sleeve 204 and a handle 206. In some embodiments, the drill sleeve 202 and guide pin sleeve 204 can be inserted within the soft tissue protector 200 to form a soft tissue protector assembly 210 that can slide over the guide pin 208 until bony contact is achieved. The soft tissue protector 200 can be any one of the soft tissue protectors or dilators or delivery sleeves disclosed herein. In some embodiments, an expandable dilator or delivery sleeve 200 as disclosed herein can be used in place of a conventional soft tissue dilator. In the case of the expandable dilator, in some embodiments, the expandable dilator can be slid over the guide pin and then expanded before the drill sleeve 202 and/or guide pin sleeve 204 are inserted within the expandable dilator. In other embodiments, insertion of the drill sleeve 202 and/or guide pin sleeve 204 within the expandable dilator can be used to expand the expandable dilator.

In some embodiments, a dilator can be used to open a channel though the tissue prior to sliding the soft tissue protector assembly 210 over the guide pin. The dilator(s) can be placed over the guide pin, using for example a plurality of sequentially larger dilators or using an expandable dilator. After the channel has been formed through the tissue, the dilator(s) can be removed and the soft tissue protector assembly can be slid over the guide pin. In some embodiments, the expandable dilator can serve as a soft tissue protector after being expanded. For example, after expansion the drill sleeve and guide pin sleeve can be inserted into the expandable dilator.

As shown in FIGS. 5 and 6, a triangular implant structure 20 can be now tapped through the soft tissue protector over the guide pin 38 through the ilium, across the SI-Joint, and into the sacrum, until the proximal end of the implant structure 20 is flush against the lateral wall of the ilium (see also FIGS. 7A and 7B). The guide pin 38 and soft tissue protector are withdrawn, leaving the implant structure 20 residing in the broached passageway, flush with the lateral wall of the ilium (see FIG. 7A and 7B). In the illustrated embodiment, two additional implant structures 20 are implanted in this manner, as FIG. 6 best shows. In other embodiments, the proximal ends of the implant structures 20 are left proud of the lateral wall of the ilium, such that they extend 1, 2, 3 or 4 mm outside of the ilium. This ensures that the implants 1020 engage the hard cortical portion of the ilium rather than just the softer cancellous portion, through which they might migrate if there was no structural support from hard cortical bone. The hard cortical bone can also bear the loads or forces typically exerted on the bone by the implant 1020.

The implant structures 20 are sized according to the local anatomy. For the SI-Joint, representative implant structures 20 can range in size, depending upon the local anatomy, from about 35 mm to about 60 mm in length, and about a 7 mm inscribed diameter (i.e. a triangle having a height of about 10.5 mm and a base of about 12 mm). The morphology of the local structures can be generally understood by medical professionals using textbooks of human skeletal anatomy along with their knowledge of the site and its disease or injury. The physician is also able to ascertain the dimensions of the implant structure 20 based upon prior analysis of the morphology of the targeted bone using, for example, plain film x-ray, fluoroscopic x-ray, or MRI or CT scanning.

Using a lateral approach, one or more implant structures 20 can be individually inserted in a minimally invasive fashion across the SI-Joint, as has been described. Conventional tissue access tools, obturators, cannulas, and/or drills can be used for this purpose. Alternatively, the novel tissue access tools described above and in co-pending U.S. Application No. 61/609,043, titled “TISSUE DILATOR AND PROTECTER” and filed Mar. 9, 2012, can also be used. No joint preparation, removal of cartilage, or scraping are required before formation of the insertion path or insertion of the implant structures 20, so a minimally invasive insertion path sized approximately at or about the maximum outer diameter of the implant structures 20 can be formed.

The implant structures 20 can obviate the need for autologous bone graft material, additional screws and/or rods, hollow modular anchorage screws, cannulated compression screws, threaded cages within the joint, or fracture fixation screws. Still, in the physician's discretion, bone graft material and other fixation instrumentation can be used in combination with the implant structures 20.

In a representative procedure, one to six, or perhaps up to eight, implant structures 20 can be used, depending on the size of the patient and the size of the implant structures 20. After installation, the patient would be advised to prevent or reduce loading of the SI-Joint while fusion occurs. This could be about a six to twelve week period or more, depending on the health of the patient and his or her adherence to post-op protocol.

The implant structures 20 make possible surgical techniques that are less invasive than traditional open surgery with no extensive soft tissue stripping. The lateral approach to the SI-Joint provides a straightforward surgical approach that complements the minimally invasive surgical techniques. The profile and design of the implant structures 20 minimize or reduce rotation and micromotion. Rigid implant structures 20 made from titanium alloy provide immediate post-op SI Joint stability. A bony in-growth region 24 comprising a porous plasma spray coating with irregular surfaces supports stable bone fixation/fusion. The implant structures 20 and surgical approaches make possible the placement of larger fusion surface areas designed to maximize post-surgical weight bearing capacity and provide a biomechanically rigorous implant designed specifically to stabilize the heavily loaded SI-Joint.

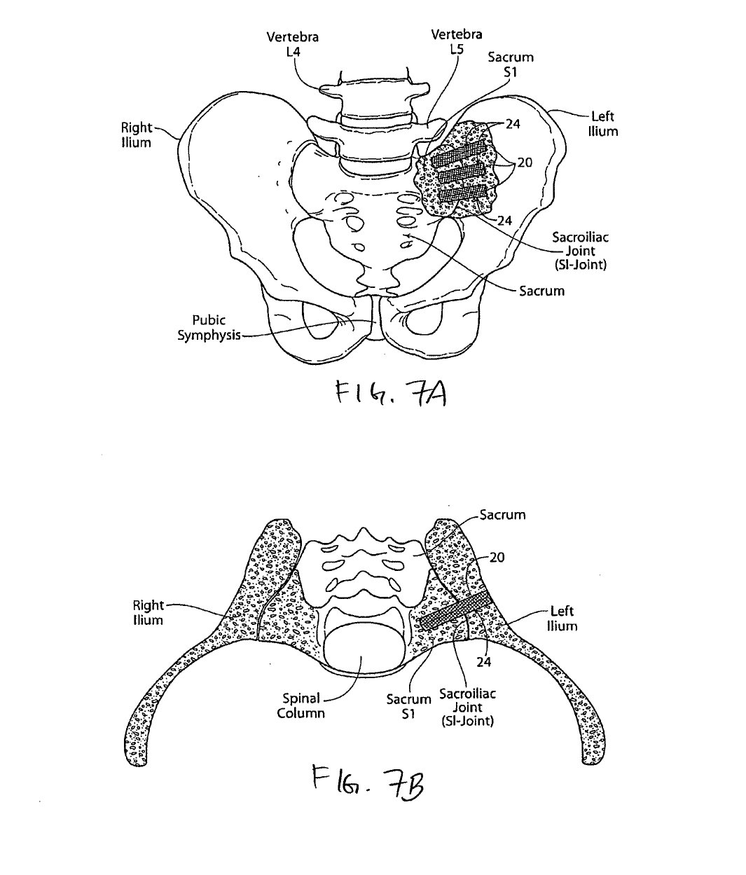

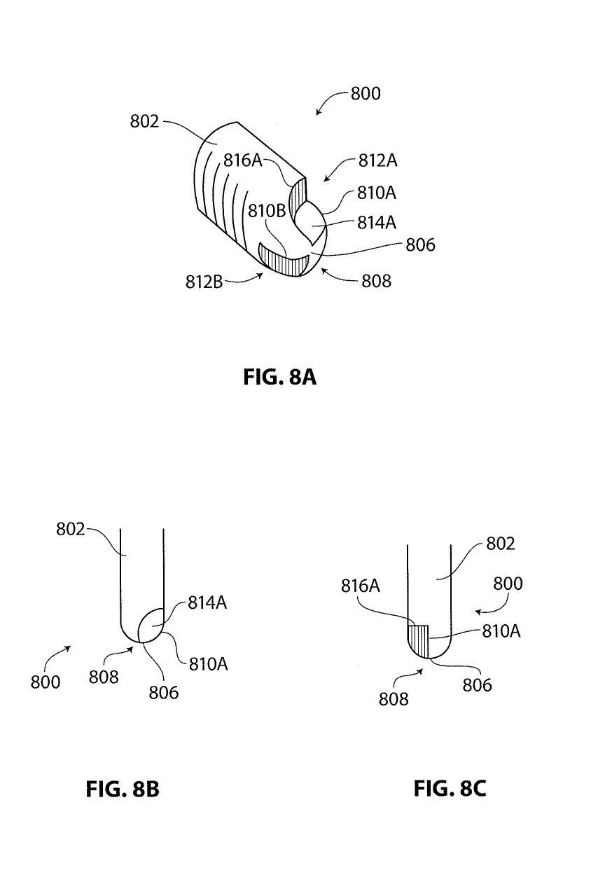

FIGS. 8A-8C illustrate one embodiment of a guide pin or wire 800, hereinafter referred to as a guide pin, which can be used with a variety of implants and surgical procedures, as, for example, described above. The guide pin 800 has an elongate body 802 with a proximal end (not shown) and a distal end 806. The distal end 806 can have a blunt or rounded tip 808 with at least one cutting edge. For example, as shown in FIGS. 8A-8C, the distal end 806 can form a ball end cutter with a substantially hemispherical or rounded tip with two cutting edges 810A and 810B. In other embodiments, the ball end cutter at the end of the guide pin 800 can have one, two, three or more cutting edges. Absent rotation, the blunt or rounded tip 808 is more difficult to simply push through bone and/or tissue when compared to a conventional pointed tip guide pin, which can be drilled, hammered and/or tapped into place. For example, when using a conventional pointed tip guide pin, striking the guide pin too hard or exerting too much force on the guide pin while drilling, especially when the tip is nearing or is already in soft tissue, can result in the guide pin advancing too far into the sacrum, potentially causing damage to the neural structures such as the L5 or Si nerve roots. In contrast, a blunt or rounded tip 808 provides more resistance to forwards motion, making it more difficult to advance the tip 808 too far by accident, thereby reducing the likelihood of inadvertently damaging neural structures during guide pin placement. Also, if the guide pin does advance, the blunt or rounded atraumatic tip will likely cause less tissue damage than a pointed tip guide pin.

The cutting edges 810A and 810B can be located on opposing sides of the ball end cutter or can be spaced evenly around the ball end cutter. The cutting edges 810A and 810B allow the guide pin 800 to be drilled into the bone in a controlled manner. In some embodiments, the elongate body 802 can include channels, threads, flutes or cut-outs 812A and 812B, hereinafter referred to as cut-outs, that extend proximally and inwardly from the cutting edges 810A and 810B. In some embodiments, the cut-out 812A can have a flat surface 814A where the outer edge of the flat surface 814A is the cutting edge 810A. In addition, the cut-out 812A can have a curved surface 816A that is joined with the flat surface 814A to form the cut-out 812A. In other embodiments, the cut-outs 812A and 812B can be helical flutes so that the guide pin 800 is effectively also a drill bit. These cut-outs 812 can serve to remove debris generated by the cutting edges 810A and 810B. In addition, some cut-outs, such as threads, can also serve to advance and or anchor the guide pin 800 in bone or tissue. In some embodiments, the cutting edges 810A, 810B only cut in a single direction. In other embodiments, the cutting edges can cut in both directions.

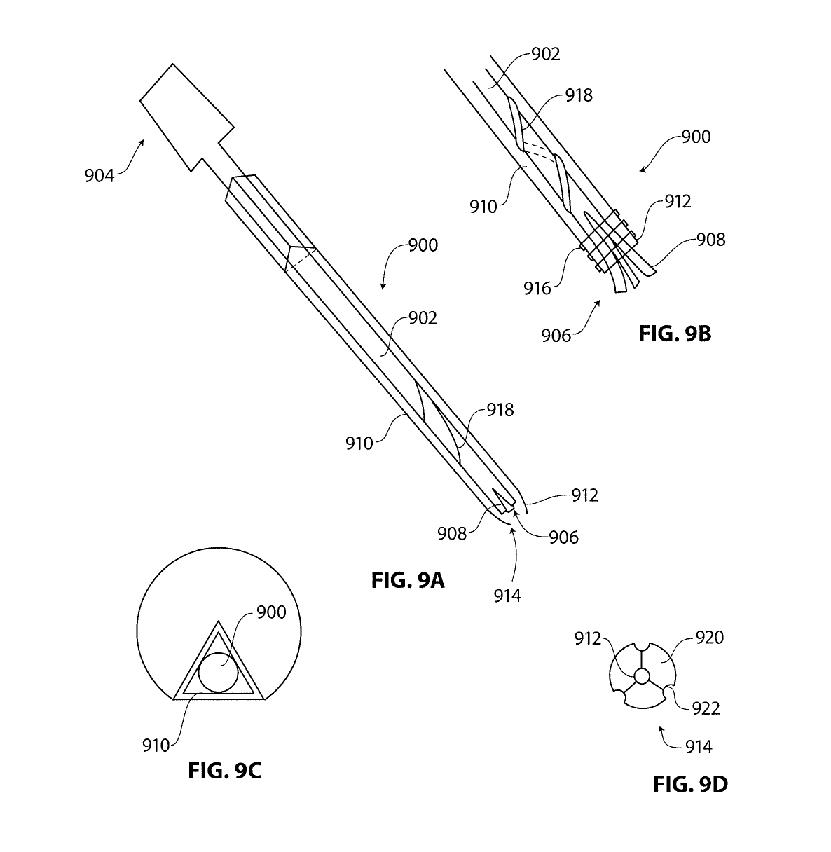

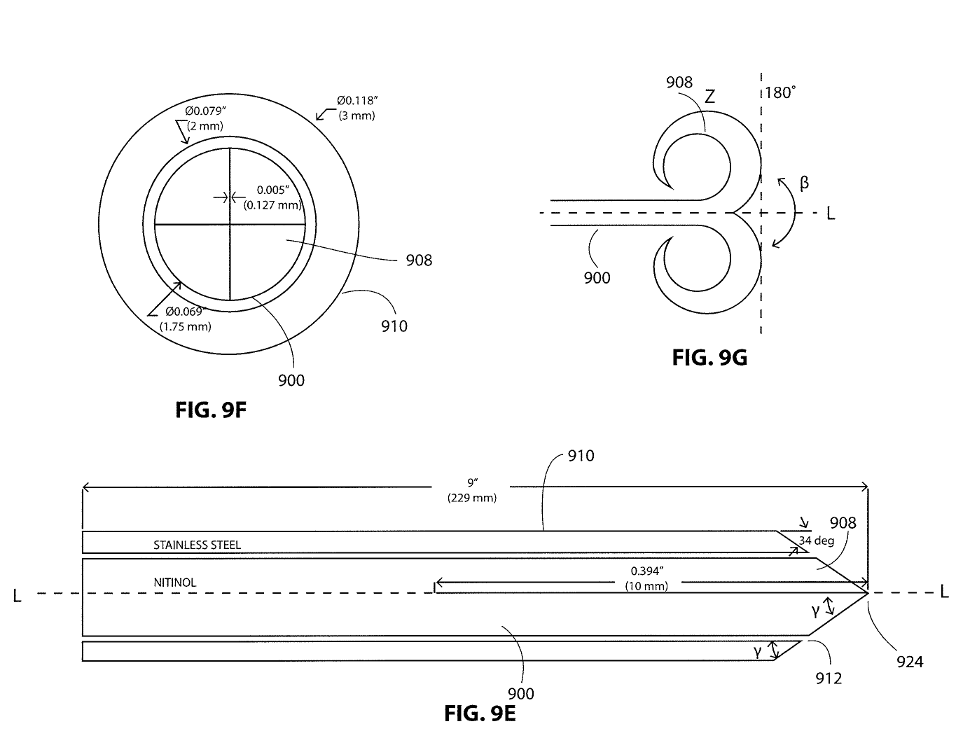

FIGS. 9A-9D illustrate another embodiment of a guide pin 900 with a feature that impedes or stops guide pin 900 advancement. The guide pin 900 has an elongate body 902 with a proximal end 904 and a distal end 906. The distal end 906 can be made of a plurality of expandable prongs 908 that can be made, along with the rest of the guide pin 900, from a variety of materials, such as metals, metal alloys, and/or shape memory metals which includes nickel titanium alloys like nitinol. Use of a shape memory metal with high elasticity, such as a nickel titanium alloy, can provide fracture resistance to the guide pin 900 and guide pin prongs 908. In some embodiments, the distal end 906 is made of at least 2, 3, or 4 or more prongs 908. The prongs 908 can have a length between about 2 mm to 20 mm, or about 5 mm to 15 mm, or about 5 mm, 10 mm, or 15 mm.

The guide pin 900 can be disposed within a guide pin sheath 910 that functions to keep the prongs 908 in a collapsed configuration. The distal end 912 of the sheath 910 can have a tapered tip 914 and threads 916 on the outer surface that facilitate advancement of the guide pin 900 through soft tissue and bone. In some embodiments, the threads 916 can have a pitch of about 1 to 32 threads per inch and a depth between about 0.25 mm to 1.5 mm. The threads 916 can also serve to anchor the sheath 910 in place within soft tissue and/or bone. The elongate body 902 of the guide pin 900 can have threads 918 with a high pitch that can advance or retract the sheath 910. Corresponding high pitch grooves may be provided on the inner surface of the sheath 910. Pin 900 may be advanced or retracted at least about 2 mm, 2 to 4 mm, 2 to 6 mm, 2 to 8 mm, or 2 to 10 mm with respect to sheath 910. In some embodiments, about a one third to one half turn of the guide pin 900 relative to the sheath 910 causes full advancement or retraction of pin 900 relative to sheath 910. Either the guide pin 900 or the sheath 910 can be rotated to advance or retract the sheath 910 from the guide pin 900. In some embodiments, the locations of the threads and the grooves may be reversed, and/or one or more laterally extending bosses or protrusions may be used instead of threads.

In some embodiments, the distal end 912 of the sheath 910 can be capable of expanding to a larger cross-sectional diameter. For example, as illustrated in FIG. 9D which is an axial view of the distal end 912 of the sheath 910, the tapered tip 914 can be made of a plurality of blade portions 920 that are capable of rotating or being displaced outwards, thereby increasing the size of the sheath opening and allowing the guide pin 900 to pass through. In some embodiments the tapered tip 914 can include stress relief portions 922 that can be a cutout or hole in the tapered tip 914 that facilitates the movement of the blade portions 920 from a non-expanded configuration to an expanded configuration. The blade portions 920 can be pushed apart into the expanded configuration by mechanical means, such as by the force exerted by the guide pin 900 as the sheath 910 is retracted. In some embodiments, the sheath 900 can be made from a metal, metal alloy, or polymer.

By retracting the sheath 910, the prongs 908 are allowed to expand to an expanded configuration. In some embodiments, the prongs 908 are made of a shape memory metal and self expand to the expanded configuration when released from the sheath 910. In other embodiments, the prongs 908 can be mechanically expanded, by for example, a mechanical actuator that forces the prongs 908 outwards. The degree of expansion can be controlled in part by the amount the sheath 910 is retracted from the prongs 908 of the guide pin 900, where the more the sheath 910 is retracted, the more the prongs 908 expand, until the prongs 908 reach full expansion. As the sheath 910 is retracted, the prongs 908 expand radially outwards, forming an anchor in the bone, particularly soft bone such as cancellous bone, and/or soft tissues. In some embodiments, in the expanded configuration, the prongs 908 can be angled at an angle a (not shown) with respect to the longitudinal axis L of the guide pin 900, where the angle a can be between about 5 to 175 degrees, or about 30 to 150 degrees, or about 45 to 135 degrees, or at least about 15, 30, 45, 60, 75, or 90 degrees.

In some embodiments, when deploying the prongs 908 within bone, cancellous bone can be chosen as the site of deployment because the prongs 908 can more easily expand within the softer material of cancellous bone. In some embodiments, when deploying the prongs 908 within harder material such as cortical bone, the prongs 908 can be at least partially deployed so that the tips of the prongs 908, which can be sharpened into points, dig into or engage the cortical bone. Expansion of the prongs 908 within bone or soft tissue increases the cross-sectional area of the guide pin 900, which increases the resistance of the guide pin 900 to further advancement, thereby preventing or impeding further advancement of the guide pin 900 and preventing or reducing the likelihood of damage to sensitive tissues such as nerve tissue.

FIGS. 9E-9G illustrate another embodiment of a guide pin 900 with prongs 908 covered by a guide pin sheath 910. In this embodiment, the guide pin 900 can have a pointed end 924 that extends distally past the distal end 912 of the sheath 910. The distal end 912 of the sheath 910 and the pointed end 924 of the guide pin 900 can be beveled or angled at an angle y with respect to the longitudinal axis L of the guide pin 900, so that the pointed end 924 of the guide pin 900 and the distal end 912 of the sheath 910 combine to form a sharp conical end capable of and/or configured to pierce soft tissue and bone. The angle y can be between about 15 degrees and 60 degrees, or between about 30 degrees and 45 degrees, or about 30, 35, 40, or 45 degrees.

In some embodiments, the sheath 910 can be made of a metal or metal alloy such as stainless steel, while the guide pin can be made of a superelastic metal or metal alloy such as a nickel titanium alloy like nitinol. In some embodiments, the length of the prongs 908 can be about 10 mm, or can be longer or shorter as described above. In some embodiments, the guide pin 900 can have a diameter between about 1 to 5 mm, or about 1, 2, 3, 4 or 5 mm, or about 1.75 mm. In some embodiments, the guide pin 900 can have 4 prongs 908, although in other embodiments, the guide pin 900 can have more or less than 4 prongs 908, as described above. In some embodiments, the four prongs 908 can be formed by two substantially perpendicular slits or cuts in the distal end of the guide pin 900 that divide the distal end into 4 substantially equal prongs 908. In some embodiments, the slits or cuts can be less than about 0.3 mm wide, or less about 0.2 mm wide, or less than 0.15 mm wide, or less than about 0.13 mm wide. Narrow slits can reduce the amount of tissue that may get trapped or embedded within the prongs 908 during insertion of the guide pin 900.

In some embodiments, the inner diameter of the sheath 910 can be about 0.1 mm, 0.15 mm, 0.2 mm, 0.25 mm, 0.3 mm, 0.35 mm, 0.4 mm, 0.45 mm, or about 0.5 mm greater than the diameter of the guide pin 900. This provides a small gap between sheath 910 and the guide pin 900 so that the guide pin 900 can freely slide within the sheath 910 while minimizing or reducing the size of the gap between two components to reduce tissue entrapment. In some embodiments, the outer diameter of the sheath 910 can be about 0.5 mm, 1.0 mm, 1.5 mm, or 2.0 mm greater than the inner diameter of the sheath 910. The sheath 910 can provide additional stiffness to the guide pin 900 and sheath 910 combination, increasing the ability of guide pin 900 and sheath 910 to penetrate soft tissue and bone. In addition, the sheath 910 has a stiffness and/or mechanical strength that can allow and/or facilitate the retraction of the expanded prongs 908 back into the sheath 910.

In some embodiments, the sheath 910 can be retracted, which allows the prongs 908 to expand into a deployed configuration. As illustrated in FIG. 9G, the prongs 908 can be angled apart at an angle β, which in some embodiments, can be greater than or equal to about 180 degrees. In other embodiments, the angle β can be greater than or equal to about 90 degrees, or 120 degrees, or 150 degrees.

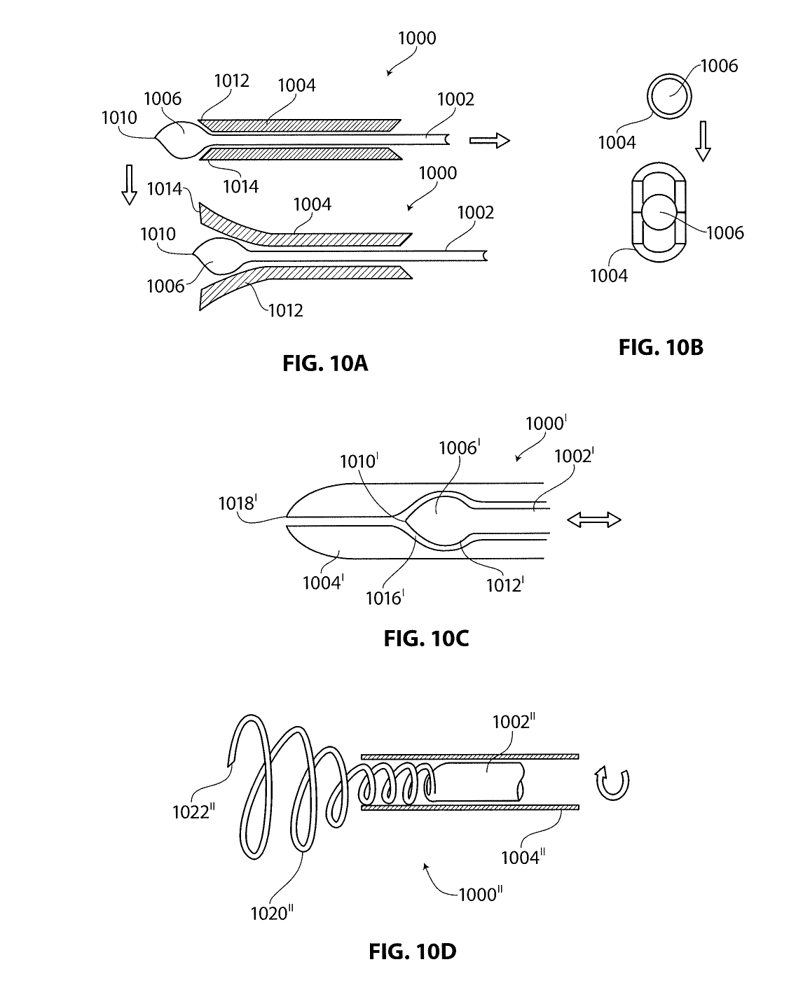

FIGS. 10A-10D illustrate other embodiments of a guide pin assembly 1000 that include a feature to reversibly stop or impede guide pin assembly 1000 advancement. For example, in one embodiment as illustrated in FIG. 10A, the guide pin assembly 1000 includes a guide pin 1002 and a guide pin sheath 1004 that covers at least the shaft 1008 of the guide pin 1002. The distal portion of the guide pin 1002 can include an expanded tip 1006 having a diameter greater than the diameter of both the guide pin shaft 1008 and the internal diameter of the sheath 1004 in a non-expanded configuration. In some embodiments, the diameter of the expanded tip 1006 can be substantially the same as the outer diameter of the sheath 1004, which provides a smooth transition from the expanded tip to the sheath. The expanded tip 1006 can have a pointed distal end 1010, which can be generally conical or formed from planes into a tetrahedral or pyramidal structure, and a tapering proximal portion 1012 that decreases in diameter until it joins the guide pin shaft 1008. The distal end of the sheath 1004 can have an inwardly tapered or beveled surface 1014 to complement the taper of the tapering proximal portion 1012 of expanded tip 1006, allowing the inwardly tapered or beveled surface 1014 to receive the tapering proximal portion 1012. The distal portion of the sheath 1004 can have a plurality of slits that allow the distal portion of the sheath 1004 to expand apart into a plurality of sections. For example, FIG. 10B illustrates an embodiment with two slits that allow the distal portion of the sheath 1004 to expand into two sections. In other embodiments, the distal portion of the sheath 1004 can have 3, 4 or more slits that divide the distal portion into 3, 4 or more expanded sections.

To expand the distal portion of the sheath 1004, the guide pin 1002 is retracted proximally, which forces the expanded tip 1006 into the channel of the sheath 1004. The tapered surfaces of both the tapering proximal portion 1012 and the inwardly tapered or beveled surface 1014 facilitate retraction of the expanded tip 1005 into the sheath 1004 by forming a ramp that the distal end of the sheath 1004 can slide over. As the guide pin 1002 is retracted proximally, the tapering proximal portion 1012 exerts an outward radial force on the inwardly tapered or beveled surface 1014 and the inner surface of the sheath 1004, resulting in the expansion of the distal end of the sheath 1004. Once the distal portion of the sheath 1004 is expanded, it has a larger cross-sectional area that resists further advancement of the guide pin assembly 1000. The expanded sections of the distal portion of the sheath 1004 can also function as anchors or barbs that resist further advancement of the guide pin assembly 1000.

To collapse the expanded sheath 1004 back to a non-expanded configuration, the guide pin 1002 can be advanced distally until the expanded tip 1006 exits the channel of the sheath 1004 and the tapered surfaces of the tapering proximal portion 1012 and the inwardly tapered or beveled surface 1014 are again aligned together. In some embodiments, the sheath 1004 is made from an elastic or superelastic material, such as a metal, metal alloy or polymer. For example, the sheath 1004 can be made of a nickel titanium alloy like nitinol.

FIG. 10C illustrates another embodiment of a guide pin assembly 1000′ that includes a feature to reversibly stop or impede guide pin assembly 1000′ advancement. In this embodiment, the guide pin 1002′ is shaped similarly to the embodiment discussed above and in FIGS. 10A and 10B, with an expanded tip 1006′ having a pointed distal end 1010′, which can be generally conical or formed from planes into a tetrahedral or pyramidal structure, and a tapering proximal portion 1012′ that decreases in diameter until it joins the guide pin shaft 1008′. However, in this embodiment in the non-expanded configuration, the expanded tip 1006′ resides within the sheath 1004′ in a recess 1016′ located within the distal portion of the sheath 1004′. The recess 1016′ can be substantially similarly shaped to the expanded tip 1006′.

The sheath 1004′ can have a pointed distal end 1018′, which can also be generally conical or formed from planes into a tetrahedral or pyramidal structure, for example, and is designed to penetrate soft tissues and/or bone. The distal portion of the sheath 1004′ can have one or more slits that divide the distal portion into at least two expandable sections that allow the distal portion of the sheath 1004′ to expand on the application of a radial outward force on the inner surface of the sheath 1004′ by the expanded tip 1006′. In this embodiment, the radial outward force that expands the distal portion of the sheath 1004′ can be generated by advancing or retracting the expanded tip 1006′ of guide pin 1002′. As the expanded tip 1006′ advances or retracts, the expanded tip 1006′ pushes apart the expandable sections of the distal portion of the sheath, thereby expanding the distal portion of the sheath. As discussed above, once the distal portion of the sheath 1004′ is expanded, it has a larger cross-sectional area that resists further advancement of the guide pin assembly 1000′. The expanded sections of the distal portion of the sheath 1004′ can also function as anchors or barbs that resist further advancement of the guide pin assembly 1000′.

To collapse the expanded sheath 1004′ back to a non-expanded configuration, the expanded tip 1006′ of the guide pin 1002′ can be advanced or retracted until the expanded tip 1006′ is seated within the recess 1016′ of the sheath 1004′. In some embodiments, the sheath 1004′ is made from an elastic or superelastic material, such as a metal, metal alloy or polymer. For example, the sheath 1004′ can be made of a nickel titanium alloy like nitinol.

FIG. 10D illustrates another embodiment of a guide pin assembly 1000″. In this embodiment, the guide pin assembly 1000″ includes a guide pin 1002″ and a sheath 1004″. The distal portion of the guide pin 1002″ can be a coiled wire 1020″ with an expanded diameter than is greater than the outer diameter of the sheath 1004″. The sheath 1004″ can have a tapered distal end as described above that in some embodiments includes at least one slit that allows the distal portion of the sheath 1004″ to expand outwards. In this embodiment, the coiled wire 1020″ is retained within the sheath 1004″ during guide pin assembly 1000″ insertion. After the guide pin assembly 1000″ is in place, the coiled wire 1020″ can be deployed by rotation of the guide pin 1002″, which advances the coiled wire 1020″ out of the sheath 1004″. The distal end 1022″ of the coiled wire 1020″ can be sharp and pointed, which allows it to penetrate through the soft tissue and/or bone while it is deployed from the sheath 1004″. As the coiled wire 1020″ exits the sheath 1004″, the coiled wire 1020″ assumes its expanded configuration, presenting a larger cross-sectional area and forming an anchor that resists further advancement of the guide pin assembly 1000″. The deployed coiled wire 1020″ can be retracted back into the sheath 1004″ by rotating the guide pin in the reverse direction. In some embodiments the coiled wire 1020″ can be made from elastic or superelastic metals or metal alloys such as a nickel titanium alloy like nitinol.

In general, the modified guide pins disclosed above can be used in place of the standard guide pins mentioned in the methods disclosed above. The modified guide pins can be inserted normally and then expanded once in place to prevent or impede further advancement of the guide pin. This feature reduces the chance of injury to sensitive tissues such as nerve tissues. In addition, although certain features have been disclosed in connection with particular embodiments, the features of one embodiment can be combined with the features of any of the other embodiments. For example, the cutting edges of one modified guide pin can be included on an expandable guide pin to create a guide pin that has both a cutting edge and the capability to expand.

In addition, the guide pins can be radiopaque so that the operator can visualize the progress of the guide pin via imaging techniques such as fluoroscopy. The guide pins or parts of the guide pins can be fabricated from radiopaque materials, or radiopaque materials can be doped, coated, or incorporated in any suitable manner with the guide pins.

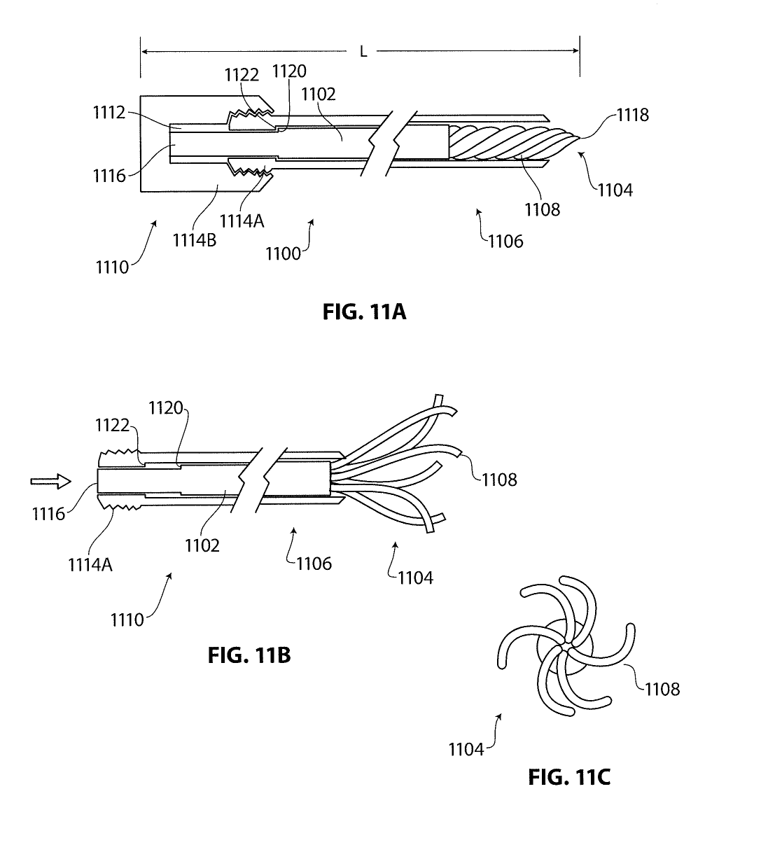

FIGS. 11A-11C illustrate another embodiment of a guide pin 1100 with an elongate shaft 1102 that includes an expandable braided wire tip 1104. A sheath 1106 covers the braided wire tip 1104, holding the strands 1108 of the braided wire tip 1104 together. The sheath 1106 can be a Jamshidi® type casing. The braided wire tip 1104 can be attached to the distal end of the elongate shaft 1102, and an impacting cap 1110 can be engaged with the proximal portion of both the elongate shaft 1102 and the sheath 1106. The impacting cap 1110 has an internal recess 1112 that can receive the proximal portion of the elongate shaft 1102 and the proximal portion of the sheath 1106. The proximal portion of the sheath 1106 can include an engagement mechanism 1114A that is complementary to an engagement mechanism 1114B within the internal recess 1112 of the impacting cap 1110. The engagement mechanisms 1114A, 1114B, which can be complementary internal and external screw threads for example, allow the impacting cap 1110 to be fastened to the sheath 1106, which shields or prevents the proximal portion of the elongate shaft 1102 from being prematurely engaged to deploy the braided wire tip 1104 out of the sheath 1106 during initial placement of the guide pin 1100 into bone.

During the insertion step, the impacting cap 1110 can be fastened to the sheath 1106 such that the proximal end 1116 of the elongate shaft 1102 abuts against a surface of the internal recess 1112 of the impacting cap 1110 and the distal tip 1118 of the braided wire tip 1104 extends out of the sheath 1106. In some embodiments, the proximal end 1116 of the elongate shaft 1102 does not abut against the surface of the internal recess 1112 but nevertheless is still disposed within the recess 1112. In some embodiments, the elongate body 1102 can have a shoulder portion 1120 that abuts against or engages with an abutment 1122 within the sheath 1106 to correctly align the elongate shaft 1102 within the sheath 1106 such that the distal tip 1118 protrudes from the sheath 1106. The distal tip 1118 of the braided wire tip 1104 can be ground or otherwise formed or molded into a trocar type point. The guide pin 1100 can be inserted into the bone in the same or similar manner as a standard guide pin. For example, the operator can strike the impacting cap 1110 and drive the pointed distal tip 1118 into the bone.

To deploy the expandable braided wire tip 1104 from the sheath 1106, the impacting cap 1110 can be removed from the sheath 1106 to expose the proximal end 1116 of the elongate shaft 1102, which can extend past the proximal end of the sheath 1106. The operator can tap on the proximal end 1116 to push the braided wire tip 1104 out of the sheath 1106 and further into the bone, where the braided wire tip 1104 can expand such that the strands 1108 are displaced outwards, thereby preventing or impeding further guide pin 1100 advancement.

In another embodiment, to deploy the expandable braided wire tip 1104 from the sheath 1106, the impacting cap 1110 can be rotated clockwise to advance the braided wire tip 1104 distal to the sheath tip and further into the bone, where the braided wire tip 1104 can expand such that the strands 1108 are displaced outwards, thereby preventing or impeding further guide pin 1100 advancement.

The braided wire tip 1104 can be formed from a plurality of strands 1108 that are twisted together in a compact configuration and are untwisted and spread apart in an expanded configuration. The strands 1108 can be made of a superelastic nickel titanium alloy, for example, that can adopt both the compact configuration and the expanded configuration. The guide pin 1100 can have a typical length and can have a diameter, including both the sheath 1106 and the elongate shaft 1102 and braided wire tip 1104 disposed within the sheath 1106, of approximately 3.2 mm, or between about 1 to 5 mm, or between about 3 to 4 mm.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A guide pin assembly, the assembly comprising:
a guide pin having a proximal end and a distal end, the distal end of the guide pin including a plurality of expandable prongs; and
a sheath having an inner surface, a tapered distal end and an opening at the distal end, the sheath configured to cover at least a portion of the guide pin;
wherein the expandable prongs are configured to be located within the sheath in a compressed state in a first configuration and are configured to be located at least partly outside the sheath in an expanded state in a second configuration.

2. The guide pin assembly of claim 1, wherein the guide pin has a shaft with threads that are engaged with corresponding grooves on the inner surface of the sheath, wherein the threads are configured to retract the sheath relative to the expandable prongs upon rotation of the guide pin relative to the sheath.

3. The guide pin assembly of claim 2, wherein the threads are pitched such that less than a half turn of the guide pin relative to the sheath is sufficient to fully deploy the expandable prongs.

4. The guide pin assembly of claim 1, wherein the guide pin is made of a shape memory metal alloy.

5. The guide pin assembly of claim 1, wherein the tapered distal end of the sheath has external threads.

6. The guide pin assembly of claim 1, wherein the tapered distal end of the sheath comprises a plurality of blade portions that are configured to be displaced outwards.

7. The guide pin assembly of claim 1, wherein the expandable prongs have a distal end that is sharpened.

8. The guide pin assembly of claim 1, wherein the distal end of the guide pin has a first beveled surface that extends past the distal end of the sheath which has a second beveled surface, wherein the first beveled surface and the second beveled surface are aligned.

9. The guide pin assembly of claim 8, wherein the first beveled surface and the second beveled surface have the same bevel angle.

10. A guide pin assembly, the assembly comprising:
a guide pin having a proximal end and a distal end and an elongate body, the distal end of the guide pin including an expanded tip; and
a sheath having an expandable distal end with at least one slit, the sheath having a channel covering at least the elongate body of the guide pin, the channel having a non-expanded configuration with a first diameter and an expanded configuration with a second diameter;
wherein the expanded tip has a diameter greater than the first diameter of the channel.

11. The guide pin assembly of claim 10, wherein the expanded tip of the guide pin extends past the distal end of the sheath when the channel is in the non-expanded configuration.

12. The guide pin assembly of claim 10, wherein the expanded tip of the guide pin is disposed within the sheath when the channel is in the non-expanded configuration.

13. The guide pin assembly of claim 10, wherein the expandable distal end of the sheath is configured to be expanded when the expanded tip of the guide pin is retracted within the channel of the sheath.

14. A guide pin assembly, the assembly comprising:
a guide pin having a proximal portion and a distal portion, the distal portion comprising a coiled wire having a distal end that is sharp and pointed; and
a sheath that is reversibly disposed over at least the distal portion of the guide pin such that the coiled wire is in a compact configuration when the sheath is disposed over distal portion and an expanded configuration when the coiled wire is advanced out of the sheath, wherein the expanded configuration of the coiled wire has a larger diameter than the sheath.

15. The guide pin assembly of claim 14, wherein the guide pin is made of a superelastic metal alloy.

16. The guide pin assembly of claim 14, wherein the guide pin is configured to be advanced and retracted relative to the sheath by rotation of the guide pin relative to the sheath.

17. A guide pin assembly, the assembly comprising:
a guide pin having a proximal portion and a distal portion, the distal portion comprising a plurality of wires that are twisted together in a compact configuration having a pointed tip; and
a sheath that is reversibly disposed over at least the distal portion of the guide pin such that the plurality of wires are in the compact configuration when the sheath is disposed over distal portion and an expanded configuration when the plurality of wires are advanced past the sheath, the sheath having a proximal end and a distal end, wherein the expanded configuration of the plurality of wires has a larger diameter than the sheath.

18. The guide pin assembly of claim 17, wherein the proximal end of the sheath is covered by a removable cap.

19. The guide pin assembly of claim 17, wherein the proximal portion of the guide pin has a smaller diameter than the distal portion such that a transverse shoulder portion provides a transition from the proximal portion to the distal portion, wherein the shoulder portion is configured to engage a complementary abutment within the sheath to align the pointed tip of the plurality of wires in the compact configuration with the distal end of the sheath.

20. A guide pin for drilling into bone, the guide pin comprising:
an elongate shaft having a proximal end and a distal end, the distal end being blunt and rounded and having at least one cut-out with a cutting edge extending proximally from the distal end.

21. The guide pin of claim 20, wherein the cut-out forms a helical or spiral flutes.

22. The guide pin of claim 20, wherein the cut-out has a flat surface that is joined with a curved surface.

23. A method of inserting a guide pin into bone, the method comprising:
drilling a guide pin through bone to a desired location in a gradual and controlled manner, the guide pin having a blunt and rounded distal end and at least one cutting edge extending from the distal end; and
viewing the location of the guide pin using fluoroscopy.
